(19) United States
(12) Patent Application Publication
(10) Pub. No.: US 2006/0130180 A1
(43) Pub. Date: Jun. 15, 2006

(54) Method of enhancing expression of exogenous polynucleotide sequences in plants

(76) Inventors: Nitzan Paldi, Doar-Na-Haela (IL); Herve Huet, Yahud (IL); Eri M. Govrin, Jerusalem (IL)

Correspondence Address: Martin Moynihan;Anthony Castorina, Suite 207, 2001 Jefferson Davis Highway, Arlington, VA, 22202, US

(21) Appl. No.: 10/542,551

(22) PCT Filed: Jan. 15, 2004
(86) PCT No.: PCT/IL04/00047

§ 371 Date: Jul. 18, 2005

(30) Foreign Application Priority Data
Jan. 16, 2003 (US) 60440362

(51) Int. Cl. A01H 1/00; C12N 7/00; C12N 15/82; C12N 15/87

(52) U.S. Cl. 800/280; 435/468; 435/235.1

(57) ABSTRACT

A method of enhancing an expression of an exogenous polynucleotide sequence in a plant which includes administering to the plant a virus selected capable of suppressing gene silencing in the plant, thereby enhancing the expression of the transgene in the plant.

### FIELD AND BACKGROUND OF THE INVENTION

The present invention relates to a method of suppressing gene silencing in plants expressing exogenous polynucleotide sequences and, more particularly, to the use of infective avirulent virus strains for enhancing expression of exogenous polynucleotide sequences in plants.

The use of genetically modified crop is growing rapidly; at present, commercially-grown transgenic crops include soybean, corn, cotton, and canola, which are cultivated in over 100 million acres worldwide. Such genetically modified commercial cultivars express exogenous polynucleotide sequences which encode herbicide tolerance, insect resistance, virus resistance, male sterility, modified color, delayed ripening and altered oil content.

In an emerging industry generically known as “molecular farming”, genetically modified plants are being used for the production of commercially valuable biomolecules such as polypeptides. Among the applications that are currently being developed in molecular farming are the production of low-cost antibodies for therapeutic and diagnostic uses, the production of copious amounts of hormones, cytokines and other bio-active molecules for the treatment of chronic or lethal diseases, the production of bio-safe substitutes for various blood components, the production of degradable plastic biopolymers, the production of unlimited amounts of processing enzymes for the food and pulp industry, the production of low-cost enzymes for waste treatments, and the production of safe bio-active molecules for the cosmetic industry.

Although useful in generating commercial amounts of such biomolecules, genetically modified plants, and in particular those that stably carry the exogenous polynucleotide sequences in their genome (also referred to as transgenic plants) suffer from one major limitation which can limit their ability to generate a commercial amount of the expressed biomolecules. Shortly after the introduction of transgenic plants, it was observed that transgene expression is at times downregulated, a phenomenon now known as “gene silencing” (Wassenegger and Pelissier, Plant Mol. Biol. 37:349-362, 1998; Napoli et al., Plant Cell 2: 279-289, 1990; van der Krol et al., Plant Cell 2:291-299, 1990). There are two kinds of known gene silencing mechanisms: transcriptional gene silencing (TGS), which results from promoter inactivation and post-transcriptional gene silencing (PTGS) which occurs when the promoter is active but the mRNA fails to accumulate (Stam et al., Annals of Botany 79: 3-12, 1997). The gene-silencing phenomenon is particularly problematic in cases where high level of transgenic expression is desired, such as the case in molecular farming. Thus, to fully exploit molecular farming potential, gene silencing needs to be controlled.

Gene silencing is believed to evolve in plants as a defense mechanism against viral infection. However, plant viruses also have evolved to avoid or suppress the host gene silencing response. Moreover, in a seemingly contradictory manner, viruses can both induce and suppress gene silencing in plants (Marathe et al., 2000; Vaucheret et al., 2001). For example, co-inoculation of a normally innocuous virus with a potyvirus (e.g., tobacco etch virus, potato virus Y) leads to substantially increasing the disease severity caused by the otherwise innocuous virus. It was further demonstrated that the co-infection synergy resulted from suppressing gene silencing by a HC protease (HC-Pro) encoded by the potyvirus (Pruss et al. 1997). Later studies reported that HC-Pro that was expressed in potyvirus-infected plants also suppressed gene silencing in transgenic plants (Anandalakshmi et al. 1998; Kasschau and Carrington, 1998; and Savenkov and Valkonen, 2001). A recent survey revealed that gene silencing may be suppressed by many different virulent viruses (Voinnet et al., 1999).

The present invention provides a novel approach for enhancing expression of exogenous polynucleotide sequences in commercial plants by inoculating the plants with a selected virus which is avirulent yet capable of suppressing gene silencing in the plants.

### SUMMARY OF THE INVENTION

According to one aspect of the present invention there is provided a method of enhancing an expression of an exogenous polynucleotide sequence in a plant. The method includes administering to the plant a virus selected capable of suppressing gene silencing in the plant, thereby enhancing the expression of the exogenous polynucleotide sequence in the plant.

According to another aspect of the present invention there is provided a method of producing a molecule of interest. The method includes administering to a plant a virus selected capable of suppressing gene silencing in the plant followed by extracting the molecule of interest being expressed in the plant, thereby producing the molecule of interest.

According to yet another aspect of the present invention there is provided a method of identifying a gene-silencing agent. The method includes inoculating a plurality of transgenic plants with a plurality of virus isolates or strains followed by selecting a plant from the infected plants which exhibits a substantially higher level of exogenous polynucleotide sequence expression than a non-infected similar transgenic plant, thereby identifying the virus isolate or strain infecting the plant as the gene silencing agent.

According to an additional aspect of the present invention there is provided an article-of-manufacturing that comprises a container including a virus selected capable of suppressing gene silencing in a plant, and a packaging material identifying the virus for use in innoculating the plant.

According to further features in preferred embodiments of the invention described below, the virus is a systemically infectious virus.

According to still further features in the described preferred embodiments the virus is an avirulent virus.

According to still further features in the described preferred embodiments the virus is a mechanically transmitted virus.

According to still further features in the described preferred embodiments the administering is effected by using an inoculation gun.

According to still further features in the described preferred embodiments the step of inoculating a plurality of transgenic plants with a plurality of virus isolates or strains further includes selecting plants which do not exhibit severe symptoms.

According to still further features in the described preferred embodiments the symptoms are selected from the group consisting of mosaic, ring spots, leaf roll, yellowing, streaking, pox formation, tumor formation, pitting and stunting.

According to still further features in the described preferred embodiments the step of inoculating a plurality of transgenic plants with a plurality of virus isolates or strains is effected by administering the virus isolates or strains suspended in a buffer solution supplemented with an abrasive material onto foliage of the transgenic plants.

According to still further features in the described preferred embodiments the exogenous polynucleotide sequence expression is quantified by an exogenous polynucleotide sequence transcribed mRNA level.

According to still further features in the described preferred embodiments the exogenous polynucleotide sequence expression is quantified by an exogenous polynucleotide sequence encoded polypeptide level.

According to still further features in the described preferred embodiments the molecule of interest is selected from the group consisting of an antibody, a vaccine, a therapeutic polypeptide, an industrial enzyme and a biopolymer.

According to still further features in the described preferred embodiments the molecule of interest is a polypeptide capable of conferring resistance or tolerance to biotic stress.

According to still further features in the described preferred embodiments the molecule of interest is a polypeptide capable of conferring resistance or tolerance to abiotic stress.

According to still further features in the described preferred embodiments the molecule of interest is a nutritionally valuable polypeptide.

According to still further features in the described preferred embodiments the virus is lyophilized.

The present invention successfully addresses the shortcomings of the presently known configurations by providing a method of enhancing expression of exogenous polynucleotide sequences in plants by infecting the plants with viruses capable of suppressing gene silencing in the plants.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a quantitative fluorometer analysis illustrating the effect of PVY strain N605 infection on the expression of an exogenous polynucleotide sequence encoded GFP in Nicotiana tabacuum. A=non-infected wild type control; B=virus-infected wild type; C=non-infected transgenic plant; D=virus-infected transgenic plant.

FIG. 2 is a quantitative Northern blot analysis illustrating the effect of CMV Banana strain infection on the expression of an exogenous polynucleotide sequence transcribed mRNA in Arabidopsis thaliana. Lane 1=non-infected wild type control (19,100); Lane 2=virus-infected transgenic plant (765,700); Lane 3=non-infected transgenic plant (320,300); Lane 4=virus-infected wild type (135,500); Lane 5=virus-infected transgenic plant (715,000).

### DESCRIPTION OF THE PREFERRED EMBODIMENTS

The present invention is of a method of enhancing expression of exogenous polynucleotide sequences in plants which method is based on infecting the plants with an avirulent virus selected capable of suppressing gene silencing in the plants.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While reducing the present invention to practice, the present inventors demonstrated for the first time that selected virus strains which are capable of systemically infecting transgenic plants without causing severe symptoms, are also capable of enhancing expression of exogenous polynucleotide sequences stably integrated into a genome of the infected plants (see Examples 1 and 2 hereinbelow for further detail).

Although the use of isolated viral sequences in downregulating gene silencing in plants and thus enhancing expression of transgenes has been previously suggested (see, U.S. Pat. Nos. 5,939,541; 6,395,962; 5,939,541 and 6,395,962), use of such isolated sequences requires transforming plants with viral components and, therefore, it is not applicable for treating existing commercial cultivars. In addition, developing new transgenic cultivars is always a laborious and time-consuming process, which may not be commercially worthwhile. Furthermore, the introduction of a viral component to plants may not necessarily mimic the effect of viral infection on gene silencing.

Thus, according to one aspect of the present invention, there is provided a method of enhancing an expression of an exogenous polynucleotide sequence in a plant by administering to the plant a virus selected capable of enhancing the expression of the exogenoius polypeptide by preferably suppressing the gene silencing mechanism in the plant.

As used herein, the term “suppressing” when used with respect to gene silencing refers to partial or complete inhibition of at least one component of the gene silencing mechanism.

As used herein, the term “exogenous polynucleotide sequence” refers to any nucleic acid sequence which does not naturally occur within the plant but which, when introduced into the plant either in a stable or transient manner, produces a polypeptide product. According to preferred embodiments of the present invention, the exogenous polynucleotide sequence is stably integrated into the plant genome in which case, the plant is typically referred to as a transgenic plant. The exogenous polynucleotide sequence can encode any type of polypeptide expressible in plants. Examples of polypeptides which are expressible in plants are provided hereinbelow.

Plant-derived polypeptides are cheaper to produce and store, easier to scale up for mass production, and safer than those derived from animals or microorganisms. Recent reviews of molecular farming include Daniell et al., (Medical molecular farming: production of antibodies, biopharmaceuticals and edible vaccines in plants, Trends Plant Sci. 6:219-226, 2001), Rishi et al. Molecular Farming in Plants: A Current Perspective, J. Plant Biochemistry and Biotechnology 10:1-12, 2001), Mor, et al. (Edible vaccines: a concept comes of age, Trends Microbiol. 6:449-453, 1998) and Tacket and Mason (A review of oral vaccination with transgenic vegetables, Microbes Infect. 1:777-783, 1999).

Polypeptides with applications in human or animal vaccines which can be expressed by transgenic plants currently include enterotoxigenic E. coli vaccine expressed in tobacco, potato and maize; Vibrio cholerae vaccine expressed in potato; Hepatitis B virus vaccine expressed in tobacco, potato, lupin and lettuce; Norwalk virus vacrine expressed in tobacco and potato; Rabies virus vaccine expressed in tomato; human cytomegalovirus vaccine expressed in tobacco; Rabbit hemorrhagic disease virus vaccine, expressed in potato; foot-and-mouth disease vaccine expressed in Arabidopsis and alfalfa; and transmissible gastroenteritis coronavirus expressed in Arabidopsis, tobacco and maize (Daniell et al., Trends Plant Sci. 6:219-226, 2001).

Antibodies expressed by transgenic plants currently include anti glycoprotein B of HSV, expressed in soybean; anti colon cancer marker antibody, expressed in tobacco; anti S. mutans (tooth decay) antibody, expressed in tobacco; Hodgkin's lymphoma ScFv of IgG from mouse, expressed in tobacco; B-cell lymphoma antibody, expressed in tobacco; anti carcinoembryogenic marker ScFV, expressed in cereals; anti human creatine kinase, expressed in Arabidopsis; anti Atrazine antibody, expressed in tobacco; and anti nematode antigen, expressed in tobacco (Rishi et al., J Plant Biochemistry and Biotechnology 10:1-12, 2001).

Polypeptides with applications in human health which can be expressed by transgenic plants currently include human protein C, expressed in tobacco; human hirudin, expressed in canola (Brassica napus); human granulocyte-macrophage colony-stimulating factor, expressed in tobacco; human somatotropin, expressed in tobacco; Nuclear expression factor, expressed in tobacco; human erythropoietin, expressed in tobacco; human enkephalins, expressed in Arabidopsis; human epidermal growth factor, expressed in tobacco; human interferon-alpha, expressed in rice, turnip (Brassica rapa); human β-interferon, expressed in tobacco; human serum albumin, expressed in tobacco; human α, β hemoglobin, expressed in tobacco; human homotrimeric collagen, expressed in tobacco; human α-1-antitrypsin, expressed in rice; human aprotinin, expressed in maize; human lactoferrin, expressed in potato; angiotensin-converting enzyme, expressed in tobacco, tomato; α-tricosanthin from TMV-U1, expressed in Nicotiana bethamiana; and glucocerebrosidase, expressed in tobacco (Daniell et al., Trends Plant Sci. 6:219-226, 2001).

Enzymes with industrial applications which can be expressed by transgenic plants currently include α-amylase, expressed in tobacco; phytase, expressed in alfalfa, tobacco; cellulose, expressed in alfalfa, potato, tobacco; manganese peroxidase, expressed in alfalfa, tobacco; β-(1,4) xylanase, expressed by tobacco, canola; β-(1,3) glugcanase, expressed by tobacco, barley; and glucuronidase, expressed in maize (Rishi et al., J Plant Biochemistry & Biotechnology Vol. 10:1-12, 2001).

Biopolymers with industrial applications that can be generated by transgenic plants currently include poly-3-hydroxyalkanoates (PHAs). PHAs show material properties that are similar to some common plastics such as polypropylene. Besides being biodegradable, PHAs are recyclable like petrochemical thermoplastics. Production of PHAs has been reported in transgenic Arabidopsis thaliana plants (Poirier et al, Science 256: 520-523, 1992), cotton (U.S. Pat. No. 5,602,321) and radish (Slater et al., Nature Biotechnology 17: 1011-1016, 1999).

A suitable exogenous polynucleotide sequence, according to the teaching of the present invention, may also encode a polypeptide capable of conferring plant resistance or tolerance to a biotic stress such as, but not limited to, pest or disease resistance. Presently known polypeptides capable of conferring pest or disease resistance in transgenic plants include, for example, insecticidal Bacillus thuringiensis (Bt) crystalline proteins, fungal cell-wall degrading enzymes such as chitinases and glugcanases (such as described in U.S. Pat. No. 6,521,4350), pathogenesis-related proteins (such as described by McDowell and Woffenden, Trends Biotechnol. 21:178-83, 2003), antimicrobial peptides (such as described in U.S. Pat. No. 6,600,090) and proteins involved in the signal transduction cascade leading to systemic acquired resistance in plants (such as described in U.S. Pat. No. 6,091,004).

A suitable exogenous polynucleotide sequence may also encode a polypeptide capable of conferring plant resistance or tolerance to an abiotic stress such as, but not limited to, drought, salinity, extreme temperature, flood, frost, malnutrition, toxic pollution, UV irradiation and a mechanical injury. Polypeptides capable of conferring plant tolerance to an abiotic stress in transgenic plants are described in, for example, U.S. Pat. Nos. 5,965,705, 6,613,919, 6,563,019 and Kasuga et al. (Nat Biotechnol. 17: 287-91, 1999).

A suitable exogenous polynucleotide sequence may also encode nutritionally valuable polypeptides such as described, for example, by Sevenier et al. (J Am Coll Nutr. 21:199-204, 2002).

As is mentioned hereinabove, infecting the plant harboring the exogenous polynucleotide sequence with a virus capable of suppressing gene silencing enhances the expression of exogenous polynucleotide sequence in the plant. There are currently over 500 known viruses capable of infecting almost all plants; examples of which are described in the web site http://image.fs.uidaho.edu/vide/. A suitable virus, according to the teaching of the present invention, is capable of suppressing gene silencing and capable of systemically infecting the plant, i.e., establishing and propagating throughout the plant body. Examples of viruses capable of systemically infecting crop plants are provided in Table 1 which follows.

TABLE 1

| Plant family | Poty- | Tobamo- | Cucumo | Nepo- | Gemini |
|---|---|---|---|---|---|
| ▾ | virus | virus | virus | virus | virus |
| Solanacea (e.g., | PVY | RMV | CMV | CGMV, | TYLCV |
| tomato, tobacco) | | | | ArMV | |
| Cereals e.g., corn, | MDMV, | RMV | | | MSV |
| wheat, rice) | SCMV | | | | |
| Soybean | SMV, | | CMV | ArMV | |
| | CMV | | | | |
| Cucurbits (e.g., | ZYMV | RMV | CMV | ArMV | SLCV |
| cucumber, melon, | | | | | |
| squash, pumkin, | | | | | |
| watermelon) | | | | | |

Abbreviations:
CMV, cucumber mosaic virus;
CGMV, cassava green mottle nepovirus;
ArMV, Arabis mosaic nepovirus;
PVY, potato virus Y;
ZYMV, zuccini yellow mosaic virus;
SMV, soybean mosaic potyvirus;
BCMV, bean common mosaic potyvirus;
MDMV, Maize dwarf mosaic potyvirus;
MSV, maize streak monogeminivirus;
SCMV, sugarcane mosaic potyvirus;
RMV, ribgrass mosaic tobamovirus;
TYLCV, tomato yellow leaf curl bigeminivirus;
SLCV, squash leaf curl virus.

Preferably, the virus is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols: From Virus Isolation to Transgenic Resistance (Methods in Molecular Biology (Humana Pr), Vol 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Isolated viruses or virus-infected sap can be freeze-dried (lyophilized) in order to improve viral preservation in storage, using methods well known in the art such as described, for example, by Rowe et al. (Cyrobiology 8: 153-72, 1971) and by Rightsel et al. (Cryobiology 3:423-31, 1967).

The virus of the present invention can be, for example, a strain or an isolate of a potyvirus (e.g., potato virus Y, PVY; tobacco etch virus, TEV), a cucumovirus (e.g., cucumber mosaic virus, CMV), a comovirus (e.g., cowpea mosaic virus, CpMV), a geminivirus (e.g., cassaya mosaic virus, ACMV), a nepovirus (e.g., nandina virus X, NMV; viola mosaic virus, VMV), a tomavirus (e.g., tobacco mosaic virus, TMV) and a tobavirus (e.g., tobacco black ring virus, TBSV).

The virus can be administered to a plant per se or as part (active ingredient) of an inoculant formulation. A suitable inoculant formulation may include the virus and an acceptable carrier such as a stabilizer.

The term “stabilizer” used herein refers to any inert substance which is capable of increasing virus stability in storage. Suitable stabilizers may include sugars such as sucrose, raffinose, glucose and trehalose, or a composition such as described, for example, in U.S. Pat. Nos. 6,290,967, 6,544,769, 4,186,195, 4,147,772, 4,000,256, 3,783,098.

Techniques for formulation and inoculation of viruses to plants may be found in Foster and Taylor, eds. “Plant Virology Protocols: From Virus Isolation to Transgenic Resistance (Methods in Molecular Biology (Humana Pr), Vol 81)”, Humana Press, 1998; Maramorosh and Koprowski, eds. “Methods in Virology” 7 vols, Academic Press, New York 1967-1984; Hill, S. A. “Methods in Plant Virology”, Blackwell, Oxford, 1984; Walkey, D. G. A. “Applied Plant Virology”, Wiley, New York, 1985; and Kado and Agrawa, eds. “Principles and Techniques in Plant Virology”, Van Nostrand-Reinhold, New York.

To facilitate use, the inoculant formulation is preferably packaged in a sealed container labeled for use as an inoculant along with information indicating suitable target plants, plant stage most suitable for inoculation and recommended application rates.

Inoculant formulations suitable for use in context of the present invention include formulations wherein the viruses are contained in an amount effective to achieve the intended purpose. For any preparation used in the methods of the invention, the effective amount or dose can be estimated initially from small-scale growth-chamber or greenhouse trials. Such information can be used to more accurately determine useful doses in commercial large-scale applications. Dosages necessary to achieve the desired effect will depend on the virus specific characteristics and the specific plant growth stage and environmental characteristic. The method of the present invention is effected as follows: A plant genetically modified to express a molecule of interest (e.g., an antibody, a vaccine, a therapeutic polypeptide an industrial enzyme, a polypeptide conferring stress resistance or tolerance, or a nutritionally valuable polypeptide) is inoculated with a suitable virus strain by applying viral containing sap or any other viral preparation to the surface of plant tissue, preferably leaves, which was previously dusted with an abrasive such as carborundum. Application of the sap is preferably made by gently rubbing the leaves with a pad dipped in the sap, with a finger, a glass spatula, a painter's brush, or with a small sprayer. In successful inoculation, the virus enters the plant cells through the wounds made by the abrasive or through other opening and initiates an infection.

For high volume inoculations, the virus is preferably administered to the plant by using a mechanized plant inoculation instrument such as described, for example, by Gal-on et al. (J. Gen. Virology 3223-3227, 1995) and in U.S. Pat. No. 6,644,341.

Once expression of the polypeptide reaches suitable levels the polypeptide may be extracted from plant tissues. General methods of extracting polypeptides from plant tissues are well known in the art such as described, for example, by Roe, S., Ed. (“Protein Purification: a Practical Approach”, Oxford University Press, 2001). Preferably, target molecules such as antibodies, vaccines and therapeutic polypeptides are extracted and purified from plant tissues using procedures such as described by Cunningham and Porter, Eds., “Recombinant Protein Production in Plants: Production and Isolation of Clinically Useful Compounds”, Humana Press, 1998; Fischer et al., Biotechnol. Appl. Biochem. 30:101-108, 1999; and Seon et al., J. Plant Biotechnology 4: 95-101, 2002. Procedures of extracting biopolymers from plant tissues are described in Doi Y. and Steinbuchel, Eds. “Biopolimers Volume 4”, Willey-VCH, 2002.

Since plants expressing a detectable transgene can be used to qualify and quantify gene silencing suppression of a specific viral isolate, the present invention also envisages a method of identifying viral isolates which are capable of effectively suppressing gene silencing in plants.

Such a method is effected by inoculating a plurality of transgenic plants expressing a detectable polypeptide (e.g., reporter polypeptide) with a plurality of virus isolates or strains, using inoculation methods such as described hereinabove. Following inoculation, an infected plant that exhibits a substantially higher level of reporter polypeptide expression than a non-infected similar transgenic plant is selected. Preferably, the selected plant does not exhibit severe symptoms such as mosaic, ring spots, leaf roll, yellowing, streaking, pox formation, tumor formation, pitting and stunting. The virus isolate or strain infecting the selected plant is thereby isolated and identified as a gene silencing suppressing agent.

Infected plants may be analyzed to determine the effect of virus inoculation on the expression of exogenous polynucleotide sequences by measuring the levels of the reporter polypeptide or its transcribed mRNAs.

Reporter polypeptide expression in plant tissues can be quantitatively analyzed using standard protein detection assays which are well known in the art such as, for example, enzyme-linked immuno-sorbent assay (ELISA) and Western blot analysis; or by using fluorescent based assays in the case of a fluorescent reporter (e.g., GFP), or a substrate based assay in the case of an enzyme reporter (e.g., luciferase). The level of transcribed mRNAs can be analyzed by hybridization-based assays such as, for example, reverse-transcription polymerase chain-reaction (RT-PCR) and Northern blot analysis (see, for example, Clark, Ed., Plant Molecular Biology: A Laboratory Manual, Springer-Verlag, Berlin, 1997; Glick and Thompson, Eds., “Methods in Plant Molecular Biology and Biochemistry, CRC Press, 1993; Dashek, W. V., Eds., “Methods in Plant Pathology and Molecular Biology, CRC Press, 1997; Weissbach and Weissbach, Methods for Plant Molecular Biology, Academic Press, 1988; and in the web site http://www.protocol-online.org/prot/Plant Biology/).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Current Protocols in Immunology” Volumes I-III Coligan J. E., Ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Increasing Expression of Transgenic GFP in Tobacco by an Avirulent PVY Strain

Materials and Methods:

Plants and growth conditions: Transgenic Nicotiana tabacum R3 and R6 carrying the Green Fluorescence Protein (GFP) gene and wild type N. tabacum SR were planted in peat-based growth mix and grown in a growth chamber under day/night conditions of 14 hr at 24° C./10 hr at 20° C.

Virus: Potato Virus Y (PVY) strain N605 described by Jakab et al. (Journal of General Virology 78:3141-3145, 1997) was used.

Virus inoculation: Tobacco leaf tissue infected with PVY N₆O₅ was ground by pestle and mortar in a 1:4 dilution of 0.05 M ice-cold phosphate buffer, pH 7.0, to produce a virus infected sap. The sap was rubbed with a cotton swab onto carborundum-dusted leaves of virus free tobacco plants.

RT-PCR analysis: Reverse Transcription Polymerase Chain Reaction (RT-PCR) was performed using SuperScript II according to the manufacturer instructions (Invitrogen Life Technologies; Huang, L. et al., Focus 22:3, 2000) using the following primers: 5′-GAT CCA GCA AAG GGG TAT TCA GCA TA (SEQ ID NO: 1) and 5′-TCT GCA TCA TGN ACR TCA GG (SEQ ID NO: 2).

Fluorometer analysis: Fluorescence specific activity was measured in plant tissue using the procedure described by Remens et al. (1999).

Results:

No visual symptoms were observed in any of the PVY infected plants, while RT-PCR analysis on the leaves 2 to 4 weeks after inoculation indicated systemic transmission of PVY within infected plants.

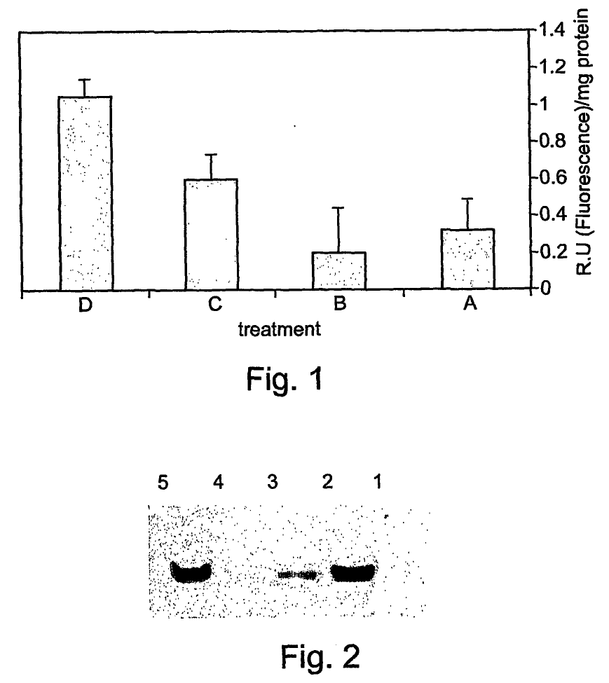

As illustrated in FIG. 1, the fluorescence specific activity measured in non-inoculated GFP-transgenic plants was 100% higher than in non-inoculated wild-type plants (0.6 and 0.3 R.U/mg protein in GFP-transgenic and wild-type plants, respectively), indicating a substantially enhanced GFP expression in the transgenic plants.

PVY infection did not alter the level of fluorescence measured in wild-type plants. However, PVY-infected GFP transgenic plants exhibited significantly higher level of fluorescence, as compared with non-infected GFP transgenic plants (1.05 and 0.6 R.U/mg protein, in PVY-infected and noninfected transgenic plants, respectively; p<0.001).

These results indicate that inoculation of transgenic tobacco plants with an avirulent PVY strain can substantially enhance the expression of exogenous polynucleotide sequences.

### Example 2

### Increasing Expression of Transgenic mRNA in Arabidopsis by an Avirulent CMV Strain

Materials and Methods:

Plants and growth conditions: Rab7 transgenic Arabidopsis thaliana and wild type A. thaliana Columbia were planted in peat-based growth mix and maintained under 16 h light at 24° C. and 8 h dark at 22° C.

Virus: Cucumber Mosaic Virus (CMV) Banana strain as described by Gafny et al. (Phytoparasitica 24:49-56, 1996) was used.

Virus inoculation: Leaf tissue of banana plants infected with CMV Banana strain was grounded by pestle and mortar in a 1:4 dilution of 0.05 M ice cold phosphate buffer, pH 7.0, to produce a virus infected sap. The sap was rubbed with a cotton swab onto carborundum-dusted leaves of virus free A. thaliana plants.

RT-PCR analysis: The PCR procedure was performed as described in example 1 above using the following primers: 5′-GAG, CGG, TCA, CAA, GAG, AGT, AG (SEQ ID NO: 3) and 5′-GGA, AAT, CAC, ACC, ACC, ACT, TA (SEQ ID NO: 4).

Northern blot analysis: RNA was blotted into Hybond N+ membrane following the protocol recommended by the manufacturer (Amersham Pharmacia Biotec).

Results:

As illustrated in FIG. 2, the inoculation of transgenic Arabidopsis thaliana with an infectious but avirulent CMV (Banana strain) resulted in 123-139% increase in the level a transgene transcribed mRNA, as compared with non-inoculated control.

These results indicate that inoculation of transgenic Arabidopsis plants with an avirulent CMV strain can substantially enhance the expression of exogenous polynucleotide sequence mRNA.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### REFERENCES

### Additional References are Cited Hereinabove

Anandalakshmi R, Pruss G. J., Ge X., Marathe R., Mallory A. C., Smith T. H., Vance V. B. (1998). A viral suppressor of gene silencing in plants. Proc. Natl. Acad. Sci. U.S.A. 95, 13079-13084
Arazi T., Slutsky S. G., Shiboleth Y. M., Wang Y., Rubinstein M., Barak S., Yang J., Gal-On A. (2001). Engineering zucchini yellow mosaic potyvirus as a non-pathogenic vector for expression of heterologous proteins in cucurbits. J. Biotechnol. 87(1):67-82.
Atreya, C. D., Raccah, B. and Pirone, T. P. (1990). A point mutation in the coat protein abolishes aphid transmissibility of a potyvirus. Virology 178, 161-165.
Atreya, C. D., Atreya, P. L. Thombury, D. W. and Pirone, T. P. (1992). Site directed mutagenesis in the potyvirus HC-Pro gene affect helper component activity, virus accumulation and symptom expression in infected tobacco plants. Virology 191, 106-11.
Baulcombe, D. Viruses and gene silencing in plants. Arch. Virol. Suppl. 15, 189-201 (1999).
Beclin C., Berthome R., Palauqui J. C., Tepfer M., Vaucheret H. (1998). Infection of tobacco or Arabidopsis plants by CMV counteracts systemic post-transcriptional silencing of non-viral (trans)genes. Virology 252:313-317.
Bernstein, E., Denli, A. M. & Hannon, G. J. The rest is silence. RNA 7, 1509-1521 (2001).
Boothe, J. G., Parmenter, D. L. & Saponja J. A. Molecular farming in plants: oilseeds as vehicles for the production of pharmaceutical proteins. Drug Develop. Res. 42, 172-181 (1997).
Brennan, F. R. et al. Chimeric plant virus particles administered nasally or orally induce systemic and mucosal immune responses in mice. J. Virol. 73, 930-938 (1999).
Gianinna Brigneti, Olivier Voinnet, Wan-Xiang Li¹, Liang-Hui Ji¹, Shou-Wei Ding¹ and David C. Baulcombe² Viral pathogenicity determinants are suppressors of transgene silencing in Nicotiana benthaniana. The EMBO Journal Vol. 17, pp. 6739-6746, 1998
Cesar L., Kasschau K. D., Carrington J. C. (2000). Virus-encoded suppressor of posttranslational gene silencing targets a maintenance step in the silencing pathway. Proc. Natl. Acad. Sci. U.S.A. 24:13401-13406.
Gal-On, A., Antignus, Y., Rosner, A. and Raccah, B. (1992). A mutation in the coat protein gene of zucchini yellow mosaic virus restored its aphid transmissibility but had no effect on its multiplication. J. Gen. Virol. 73, 2183-2187.
Gal-On A, and Raccah B, (2000). A point mutation in the FRNK motif of the potyvirus HC-pro gene alters the symptom expression in cucurbits and exhibits protection against severe homologous virus. Phytopath. 90,1056.
Gonsalves, D. and Garnsey, S. M. (1989) Cross-protection techniques for control of plant virus diseases in the Tropics. Plant Disease 73, 592-597.
Guo H. S., Lopez-Moya J. J., Garcia J. A. (1998). Susceptibility to recombination rearrangements of a chimeric plum pox potyvirus genome after insertion of a foreign gene. Virus Res. 57(2):183-95.
Hamilton, A. J., Lycett, G. W. & Grierson, D. Antisense gene that inhibits synthesis of the hormone ethylene in transgenic plants. Nature 346, 284-287 (1990).
Hamilton, A. J. & Baulcombe, D. C. A species of small antisense RNA in posttranscriptional gene silencing in plants. Science 286, 950-952 (1999).
Huet, H., Gal-On, A., Meir, E. Lecoq, H. and Raccah, B. (1994). Mutations in the helper component (HC) gene of zucchini yellow mosaic virus (ZYMV) affect aphid transmissibility. J. Gen. Virol. 75, 1407-1414.
L. H. Ji, S. W. Ding, Mol. Plant-Microbe Interact., in press Johnson, E. Edible plant vaccines. Nat. Biotechnol. 14,1532-1533 (1996).
Kasschau, K. D. And carrington, J. C. (1998). A counter defensive strategy of plant viruses: suppression of post transcriptional gene silencing. Cell 95: 461-470
Kumagai, M. H. et al. Rapid, high-level expression of biologically active alpha-trichosanthin in transfected plants by an RNA viral vector. Proc. Natl. Acad. Sci. USA 90, 427-430 (1993).
Lindbo J. A., Fitzmaurice W. P., Della-Cioppa G. (2001). Virus-mediated reprogramming of gene expression in plants. Curr Opin Plant Biol. 4(3): 181-5.
Mallory A. C., Ely L., Smith T. H., Marathe R., Anandalakshmi R., Fagard M., Vaucheret H., Pruss G., Bowman L., Vance V. B. (2001). HC-Pro suppression of transgene silencing eliminates small RNAs but not transgene methylation or the mobile signal. Plant Cell 13:571-583.
Marathe R, Anandalakshmi R., Smith T. H., Pruss G. J., Vance V. B. (2000). RNA viruses as inducers, suppressors and targets of post-transcriptional gene silencing. Plant Mol. Biol. 43(2-3):295-306.
Martienssen, R. A. & Colot, V. DNA methylation and epigenetic inheritance in plants and filamentous fungi. Science 293, 1070-1074 (2001).
Matthews, R. E. F. (1991) Plant Virology, 3rd edn. Academic Press Inc., San Diego, 835 pp.
McCormick A. A. et al. Rapid production of specific vaccines for lymphoma by expression of the tumor-derived single-chain Fv epitopes in tobacco plants. Proc. Natl. Acad. Sci. USA 96, 703-708 (1999).
McGarvey, P. B. et al. Expression of the rabies virus glycoprotein in transgenic tomatoes. Bio/Technology 13, 1484-1487 (1995).
McKinney, H. H. (1929) Mosaic diseases in the Canary Islands, West Africa and Gibraltar. Journal of Agricultural Research 39, 557-578.
Moloney, M. M. “Molecular farming” in plants: achievements and prospects. Biotechnol. Eng. 9, 3-9 (1995).
Napoli, C. A., Lemieux, C., & Jorgensen, R. Introduction of a chimeric chalcone synthetase gene in Petunia results in reversible cosuppression of homologous genes in trans. Plant Cell 2, 279-289 (1990).
Palauqui, J. C., Elmayan, T., Pollien, J. M. & Vaucheret, H. Systemic acquired silencing: transgene-specific post-transcriptional silencing is transmitted by grafting from silenced stocks to non-silenced scions. EMBO J. 16, 4738-4745 (1997).
Powell-Abel, P. et al. Delay of disease development in transgenic plants that express the tobacco mosaic virus coat protein gene. Science 232, 738-743 (1986).
Pruss G., Ge X., Shi X. M., Carrington J. C., Bowman Vance V. (1997). Plant viral synergism: the potyviral genome encodes a broad-range pathogenicity enhancer that transactivates replication of heterologous viruses. Plant Cell 9: 859-868.
Ratcliff F., Martin-Hernandez A. M, Baulcombe D. C. (2001). Tobacco rattle virus as a vector for analysis of gene function by silencing. Plant J. 25(2):237-45.
Remans T., Schenk P. M., Manners J. M., Grof C. P. L. Elliott A. R. (1999). A protocol for Fluorometric Qantification of mGFP5-ER and sGFP(S65T) in Transgenic Plants. Plant Molecular Biology Reporter 17:385-395.
Revers, F., LeGall, O., Candresse, T. and Maule A. J. (1999). New advances in understanding the molecular biology of plant/potyvirus interactions. Mol. Plant Microbe Interact. 12, 367-376.
Ruiz, M. T., Voinnet, O. & Baulcombe, D. C. Initiation and maintenance of virus-induced gene silencing. Plant Cell 10, 937-946 (1998).
Savenkov E I, Valkonen J P. (2001). Coat protein gene-mediated resistance to Potato virus A in transgenic plants is suppressed following infection with another potyvirus. J Gen Virol 82:2275-2278.
Schiebel, W. et al. Isolation of an RNA-directed RNA polymerase-specific cDNA clone from tomato. Plant Cell 10, 2087-2101 (1998).
Silhavy D, Molnar A, Lucioli A, Szittya C, Homyik C, Tavazza M, Burgyan J. (2002). A viral protein suppresses RNA silencing and binds silencing-generated, 21- to 25-nucleotide double-stranded RNAs. EMBO 21:3070-80.
Vaucheret H., Beclin C., Fagard M. (2001). Post-transcriptional gene silencing in plants. J. Cell. Sci. 114:3083-3091.
Voinnet, O. Pinto Y. M., Baulcombe D. (1999). Suppression of gene silencing: a general strategy used by diverse DNA and RNA viruses of plants. Proc. Natl. Acad. Sci. U.S.A. 96:14147-14152.
Voinnet, O., Ledherer C., Baulcombe D. (2000). A viral movement protein prevents systemic spread of the gene silencing signal in Nicotiana benthamiana. Cell 103:157-167.
Voinnet O. (2001). RNA silencing as a plant immune response against plant viruses. Trends In Genetics 17: 449-459.
Waterhouse P. M., Wang M., Lough T. (2001). Gene silencing as an adaptive defense against viruses Nature 411: 834-842.

What is claimed is:

1. A method of enhancing an expression of an exogenous polynucleotide sequence in a plant, comprising administering to the plant a virus selected capable of suppressing gene silencing in said plant, thereby enhancing the expression of the exogenous polynucleotide sequence in said plant.

2. The method of claim 1, wherein said virus is a systemically infectious virus.

3. The method of claim 1, wherein said virus is an avirulent virus.

4. The method of claim 1, wherein said virus is a mechanically transmitted virus.

5. The method of claim 1, wherein said administering is effected by using an inoculation gun.

6. A method of identifying a gene silencing agent, comprising:
(a) inoculating a plurality of transgenic plants with a plurality of virus isolates or strains thereby generating a plurality of infected plants; and
(b) selecting a plant from said infected plants which exhibits a substantially higher level of exogenous polynucleotide sequence expression than a non-infected similar transgenic plant, thereby identifying the virus isolate or strain infecting said plant as the gene silencing agent.

7. The method of claim 6, wherein step (a) further includes selecting plants which do not exhibit severe symptoms.

8. The method of claim 6, wherein step (a) is effected by administering said virus isolates or strains suspended in a buffer solution supplemented with an abrasive material onto foliage of said transgenic plants.

9. The method of claim 6, wherein said symptoms are selected from the group consisting of mosaic, ring spots, leaf roll, yellowing, streaking, pox formation, tumor formation, pitting and stunting.

10. The method of claim 6, wherein said exogenous polynucleotide sequence expression is quantified by an exogenous polynucleotide sequence transcribed mRNA level.

11. The method of claim 6, wherein said exogenous polynucleotide sequence expression is quantified by said exogenous polynucleotide sequence encoded polypeptide level.

12. A method of producing a molecule of interest, comprising:
(a) administering to a plant a virus selected capable of suppressing gene silencing in said plant; and
(b) extracting the molecule of interest being expressed in said plant, thereby producing said molecule of interest.

13. The method of claim 12, wherein said molecule of interest is selected from the group consisting of an antibody, a vaccine, a therapeutic polypeptide, an industrial enzyme and a biopolymer.

14. The method of claim 12, wherein said molecule of interest is a polypeptide capable of conferring resistance or tolerance to biotic stress.

15. The method of claim 12, wherein said molecule of interest is a polypeptide capable of conferring resistance or tolerance to abiotic stress.

16. The method of claim 12, wherein said molecule of interest is a nutritionally valuable polypeptide.

17. The method of claim 12, wherein said virus is a systemically infectious virus.

18. The method of claim 1, wherein said virus is an avirulent virus.

19. The method of claim 12, wherein said virus is a mechanically transmitted virus.

20. The method of claim 12, wherein said administering is effected by using an inoculation gun.

21. An article-of-manufacturing, comprising a container including a virus selected capable of suppressing gene silencing in a plant, and a packaging material identifying said virus for use in innoculating said plant.

22. The method of claim 21, wherein said virus is a systemically infectious virus.

23. The method of claim 21, wherein said virus is an avirulent virus.

24. The method of claim 21, wherein said virus is a mechanically transmitted virus.

25. The method of claim 21, wherein said virus is lyophilized.


SEQUENCE LISTING (4 sequences)

SEQ ID NO: 1 - DNA, 26 bp, Artificial sequence
  Single strand DNA oligonucleotide
gatccagcaaaggggtattcagcata

SEQ ID NO: 2 - DNA, 20 bp, Artificial sequence
  Single strand DNA oligonucleotide
tctgcatcatgnacrtcagg

SEQ ID NO: 3 - DNA, 20 bp, Artificial sequence
  Single strand DNA oligonucleotide
gagcggtcacaagagagtag

SEQ ID NO: 4 - DNA, 20 bp, Artificial sequence
  Single strand DNA oligonucleotide
ggaaatcacaccaccactta